(19) United States
(12) Patent Application Publication
(10) Pub. No.: US 2025/0073296 A1
(43) Pub. Date: Mar. 6, 2025

(54) CHINESE HERBAL MEDICINE EXTRACT, METHOD FOR PREPARING THE SAME, AND USE OF THE SAME

(71) Applicant: CHI DON BIOTECHNOLOGY CO.,LTD., Hsinchu City (TW)

(72) Inventors: MING-TANG TSENG, Hsinchu City (TW); SHU-CHING WEN, Hsinchu City (TW); HSIAO-CHUN TSENG, Hsinchu City (TW); SHIH-CHANG HSU, Hsinchu City (TW); KUO-HO WEN, Hsinchu City (TW); TZU-HAO TSENG, Hsinchu City (TW); YI-CHEN WANG, Hsinchu City (TW); JIN-KUEI WONG, Hsinchu City (TW); TZA-ZEN CHAUNG, Hsinchu City (TW); SHAU-KU HUANG

(73) Assignee: CHI DON BIOTECHNOLOGY CO.,LTD., Hsinchu City (TW)

(21) Appl. No.: 18/725,674

(22) PCT Filed: Oct. 13, 2022
(86) PCT No.: PCT/CN2022/124991

§ 371 Date: Jun. 28, 2024

(30) Foreign Application Priority Data
Dec. 29, 2021 (CN) 202111635548.X

(51) Int. Cl. A61K 36/54; A61K 36/483; A61K 36/835

(52) U.S. Cl. CPC A61K 36/54; A61K 36/483; A61K 36/835; A61K 2236/331; A61K 2236/37; A61K 2236/51; A61K 2236/53

(57) ABSTRACT

A Chinese herbal medicine extract, a method for preparing the same, and a use of the same are disclosed. The Chinese herbal medicine extract includes an active ingredient, which contains any one of or any combination of agarwood, Chinese honeylocust fruit, Chinese honeylocust spine, cinnamon leaf, and camphor leaf. Medication based on the Chinese herbal medicine extract is useful in treating coronavirus-related symptoms or diseases.

### BACKGROUND OF THE INVENTION

### 1. Technical Field

The present invention relates to a Chinese herbal medicine extract, and more particularly to a Chinese herbal medicine extract, a method for preparing the same, and a use of the same.

### 2. Description of Related Art

Coronaviruses are a group of zoonotic RNA viruses that causes diseases in humans and animals. They are spherical or oval-shaped under an electron microscope. Coronaviruses are named for the crown-like cystic fibrillar collagen protrusions on their surface. A coronavirus genome contains about 26,000 to 32,000 base pairs, making them one of the largest RNA viruses.

The novel coronavirus SARS-COV-2 (initially reported as COVID-19, and later formally denominated as SARS-COV-2 by the Coronavirus Study Group of the International Committee on Taxonomy of Viruses according to the phylogenetic relationship, taxonomy, and conventions) is a new virus strain. SARS-COV-2 binds to receptors at the human cell surface with its spike proteins, and uses 3CLpro protease cut from the protein chains produced by transcription of viral genes to generate major proteins of the virus, thereby achieving infection and replication of the virus.

With the spread of the coronavirus pandemic, more and more infected people have suffered from classic symptoms of the coronavirus, including respiratory conditions, fever, coughs, tachypnea, and dyspnea, and even death in the worst case. Hence, there is a pressing need for a medication effective in treating coronavirus-related symptoms or diseases.

### SUMMARY OF THE INVENTION

In order to address the foregoing issues, the present invention provides a Chinese herbal medicine extract, a method for preparing the same, and a use of the same. Medication based on the Chinese herbal medicine extract is useful in treating coronavirus-related symptoms or diseases.

The present invention provides a Chinese herbal medicine extract, which contains an active ingredient. The active ingredient contains any one of or any combination of agarwood, Chinese honeylocust fruit, Chinese honeylocust spine, cinnamon leaf, and camphor leaf.

In another embodiment of the present invention, the active ingredient contains agarwood, Chinese honeylocust fruit, Chinese honeylocust spine, cinnamon leaf, and camphor leaf, and the weight ratio of agarwood, Chinese honeylocust fruit, Chinese honeylocust spine, cinnamon leaf, and camphor leaf is 1˜5:1˜5:1˜5:1˜5:1˜5.

In another embodiment of the present invention, the weight ratio of agarwood, Chinese honeylocust fruit, Chinese honeylocust spine, cinnamon leaf, and camphor leaf is 1:1:1:1:1.

Another embodiment of the present invention provides a method for preparing a Chinese herbal medicine extract. The method comprises steps of: extraction: mixing a raw material containing any one of or any combination of agarwood, Chinese honeylocust fruit, Chinese honeylocust spine, cinnamon leaf, and camphor leaf with ultrapure water in a weight ratio of between 1:5 and 1:10 to form a mixture, and extracting the mixture under heating to obtain a liquid extract, in which the mixture is extracted at an extraction temperature of between 90° C. and 110° C. for an extraction period of between 1.5 hours and 2.5 hours; filtration: filtering the liquid extract; and dehydration: freeze drying the liquid extract under a vacuum to obtain the Chinese herbal medicine extract.

In another embodiment of the present invention, the raw material contains agarwood, Chinese honeylocust fruit, Chinese honeylocust spine, cinnamon leaf, and camphor leaf, and the weight ratio of agarwood, Chinese honeylocust fruit, Chinese honeylocust spine, cinnamon leaf, and camphor leaf is 1˜5:1˜5:1˜5:1˜5:1˜5.

In another embodiment of the present invention, the weight ratio of agarwood, Chinese honeylocust fruit, Chinese honeylocust spine, cinnamon leaf, and camphor leaf is 1:1:1:1:1.

Another embodiment of the present invention provides a use of the Chinese herbal medicine extract for manufacturing a medication for treating a coronavirus-related symptom or disease, and the active ingredient of the Chinese herbal medicine extract is cinnamon leaf.

Another embodiment of the present invention provides a use of the Chinese herbal medicine extract for manufacturing a medication for intervening spike proteins of SARS-COV-2, and the active ingredient of the Chinese herbal medicine extract is cinnamon leaf.

Another embodiment of the present invention provides a use of the Chinese herbal medicine extract for manufacturing a medication for inhibiting activity of SARS-CoV-2 3CLpro protease, and the active ingredient of the Chinese herbal medicine extract is cinnamon leaf.

In another embodiment of the present invention, the medication has a half-maximal inhibitory concentration IC50 of 10˜15 μg/mL.

Another embodiment of the present invention provides a use of the Chinese herbal medicine extract for manufacturing a medication for treating a coronavirus-related symptom or disease.

Another embodiment of the present invention provides a use of the Chinese herbal medicine extract for manufacturing a medication for intervening spike proteins of SARS-COV-2.

In another embodiment of the present invention, the medication has a half-maximal effective concentration EC50 of 20˜30 μg/mL.

Another embodiment of the present invention provides a use of the Chinese herbal medicine extract for manufacturing a medication for inhibiting activity of SARS-CoV-2 3CLpro protease.

In another embodiment of the present invention, the medication has a half-maximal inhibitory concentration IC50 of 26˜40 μg/mL.

In another embodiment of the present invention, for the use of the Chinese herbal medicine extract, the medication is formulated as a mouthwash, an oral/nasal inhaler, or an oral capsule.

Thereby, administration of the medication containing the Chinese herbal medicine extract effectively intervenes binding of spike proteins of coronavirus to hACE2 (Human Angiotensin-converting Enzyme 2) at human cell surface and inhibits activity of 3CLpro protease of coronavirus, so as to achieve treatment of coronavirus-related symptoms or diseases.

### BRIEF DESCRIPTION OF THE DRAWINGS

FIG. 1 is a flowchart of preparation of the Chinese herbal medicine extract of the present invention.

FIG. 2 shows analysis of intervention of the Chinese herbal medicine extract of the first embodiment of the present invention in binding of SARS-COV-2 spike proteins to in-vitro synthesized hACE2.

FIG. 3 shows analysis of the half-maximal inhibitory concentration IC50 of the Chinese herbal medicine extract of the first embodiment of the present invention with respect to binding of SARS-COV-2 spike proteins to in-vitro synthesized hACE2.

FIG. 4 shows analysis of the half-maximal effective concentration EC50 of the Chinese herbal medicine extract of the first embodiment of the present invention with respect to binding of SARS-COV-2 pseudovirus to SmBiT-ACE2 expressing cells.

FIG. 5 shows analysis of the inhibiting effect of the Chinese herbal medicine extract of the first embodiment of the present invention on the activity of SARS-CoV-2 3CLpro protease.

FIG. 6 shows analysis of the half-maximal inhibitory concentration IC50 of the Chinese herbal medicine extract of the first embodiment of the present invention for inhibiting the activity of SARS-CoV-2 3CLpro protease.

FIG. 7 shows analysis of the half-maximal inhibitory concentration IC50 of the Chinese herbal medicine extract of the second embodiment of the present invention for inhibiting the activity of SARS-CoV-2 3CLpro protease.

Therein, S1: Extraction; S2: Filtration; S3: Dehydration.

### DETAILED DESCRIPTION OF THE INVENTION

For further illustrating the means and functions by which the present invention achieves the certain objectives, the following description, in conjunction with the accompanying drawings and preferred embodiments, is set forth as below to illustrate the implement, structure, features and effects of the subject matter of the present invention.

The present invention provides a Chinese herbal medicine extract and a method for preparing the same. The Chinese herbal medicine extract contains an active ingredient. The active ingredient contains any one of or any combination of agarwood, Chinese honeylocust fruit, Chinese honeylocust spine, cinnamon leaf, and camphor leaf.

In one embodiment of the present invention, the active ingredient contains a simple-formula Chinese herbal medicine of any of agarwood, Chinese honeylocust fruit, Chinese honeylocust spine, cinnamon leaf, and camphor leaf.

In one embodiment of the present invention, the active ingredient is a complex-formula Chinese herbal medicine of any two of agarwood, Chinese honeylocust fruit, Chinese honeylocust spine, cinnamon leaf, and camphor leaf, and is selected from the group consisting of the combination of agarwood and Chinese honeylocust fruit, the combination of agarwood and Chinese honeylocust spine, the combination of agarwood and cinnamon leaf, the combination of agarwood and camphor leaf, the combination of Chinese honeylocust fruit and Chinese honeylocust spine, the combination of Chinese honeylocust fruit and cinnamon leaf, the combination of Chinese honeylocust fruit and camphor leaf, the combination of Chinese honeylocust spine and cinnamon leaf, the combination of Chinese honeylocust spine and camphor leaf, and the combination of cinnamon leaf and camphor leaf.

In one embodiment of the present invention, the active ingredient is a complex-formula Chinese herbal medicine of any three of agarwood, Chinese honeylocust fruit, Chinese honeylocust spine, cinnamon leaf, and camphor leaf, such as the combination of agarwood and Chinese honeylocust fruit and Chinese honeylocust spine, the combination of agarwood and Chinese honeylocust fruit and cinnamon leaf, or the combination of agarwood and Chinese honeylocust fruit and camphor leaf.

In one embodiment of the present invention, the active ingredient is a complex-formula Chinese herbal medicine of any four of agarwood, Chinese honeylocust fruit, Chinese honeylocust spine, cinnamon leaf, and camphor leaf, such as the combination of agarwood and Chinese honeylocust fruit and Chinese honeylocust spine and cinnamon leaf, or the combination of Chinese honeylocust fruit and Chinese honeylocust spine and cinnamon leaf and camphor leaf.

In one embodiment of the present invention, the active ingredient contains agarwood, Chinese honeylocust fruit, Chinese honeylocust spine, cinnamon leaf, and camphor leaf. The weight ratio of agarwood, Chinese honeylocust fruit, Chinese honeylocust spine, cinnamon leaf and camphor leaf is preferably 1˜5:1˜5:1˜5:1˜5:1˜5. In the present embodiment, the weight ratio of agarwood, Chinese honeylocust fruit, Chinese honeylocust spine, cinnamon leaf and camphor leaf is 1:1:1:1:1.

### Materials and Processing

### 1. Materials

### (a) Agarwood

Agarwood is derived from resin-containing timber of Incense Tree, which is a dicotyledon in the family Thymelaeaceae. Agarwood is known for its capability of regulating qi-flow for relieving pain, downbearing counterflow for regulating spleen and stomach, warming kidney for improving qi reception, dredging channels for tranquillization, and sedation as well as analgesia. In addition, according to pharmacological research, agarwood pharmacologically has significant anti-bacterial effects. For the purpose of the present embodiment, the material of agarwood may be Aquilaria sinensis from Vietnam or Taiwan. In other possible embodiments, the material of agarwood may be any of Aquilaria crassna, Aquilaria malaccensis, and Aquilaria yunnanensis.

### (b) Chinese Honeylocust Fruit

Chinese honeylocust fruit is derived from Gleditsia sinensis in the family Fabaceae, and is actually the ripe pod of Gleditsia sinensis. Chinese honeylocust fruit is effective in dispelling stubborn phlegm, breaking block and freeing orifices, relieving rheumatic pains, and destroying parasites. Chinese honeylocust fruit contains Triacanthin, and is pharmacologically proven to be inhibiting to germs.

### (c) Chinese Honeylocust Spine

Chinese honeylocust spine is derived from Gleditsia sinensis in the family Fabaceae, and is actually the spine of Gleditsia sinensis. Chinese honeylocust spine is effective in dispersing swelling, resolving toxin, expelling pus, and destroying parasites. From Chinese honeylocust spine, flavonoids, phenols, and amino acid can be extracted. Chinese honeylocust spine extract is pharmacologically proven to be bacteriostatic and disinfectant.

### (d) Cinnamon Leaf

Cinnamon leaf is derived from Cinnamomum osmophloeum in the family Lauraceae, and is effective in dissipating cold and relieving pain, promoting blood circulation, and fortifying stomach. Cinnamon leaf extract is pharmacologically proven to be effective in inhibiting bacterial growth and thereby being bacteriostatic and disinfectant.

### (e) Camphor Leaf

Camphor leaf is derived from Cinnamomum camphora in the family Lauraceae, and is effective in relieving rheumatic pains, eliminating dampness, resolving toxin, and destroying parasites. Camphor leaf extract is pharmacologically proven to be bacteriostatic and disinfectant.

### 2. Processing of Materials

(a) Agarwood, Chinese honeylocust fruit, Chinese honeylocust spine, cinnamon leaf, and camphor leaf are washed and dried in the shade overnight, separately.

(b) Agarwood, Chinese honeylocust fruit, Chinese honeylocust spine, cinnamon leaf, and camphor leaf are then oven-baked to a moisture content of preferably 5%˜20%, and more preferably 10%.

(c) Agarwood, Chinese honeylocust fruit, Chinese honeylocust spine, cinnamon leaf, and camphor leaf are crushed in a crusher separately to a particle size of preferably 10˜30 mesh, and more preferably 20 mesh.

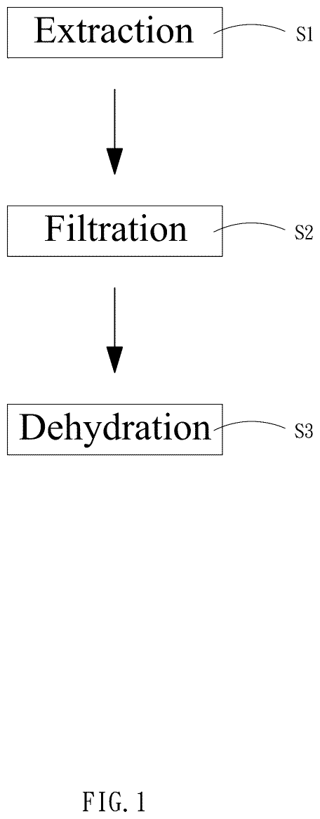

Referring to FIG. 1, to prepare the Chinese herbal medicine extract of the present invention, the following steps are involved.

First is extraction S1. Therein, a raw material containing any one of or any combination of agarwood, Chinese honeylocust fruit, Chinese honeylocust spine, cinnamon leaf, and camphor leaf is mixed with ultrapure water in a weight ratio of between 1:5 and 1:10, and preferably 1:10. The mixture of the raw material and ultrapure water is frozen in a liquid nitrogen tank and then molten at the room temperature. Afterward, cycles of freezing and thawing are performed to break cell walls of the raw material. The mixture is then extracted under heating to produce a liquid extract. Therein, the extraction temperature is of preferably 90° C.˜110° C., and more preferably of 100° C., and the extraction time is preferably 1.5 hours˜2.5 hours, and more preferably 2 hours.

The second step is filtration S2. Particularly, the liquid extract is cooled and filtered. In the present embodiment, the liquid extract is filtered through 4 stages. The liquid extract is first roughly filtered. The roughly filtered liquid extract is then processed in a centrifuge to obtain the supernatant. The centrifugal processing may be conducted at 7,000˜15,000 rpm for 3˜10 minutes, and preferably 10,000 rpm for 5 minutes. The supernatant is then filtered using qualitative filter paper (NO. 4, Whatman) of 15˜30 μm and glass fiber membrane filter (MGC) of a smaller size, like 1.2 μm, successively, so as to produce well filtered liquid extract.

The third step is dehydration S3. After filtration, the filtered liquid extract is frozen at −35˜−10° C. and then subject to freeze drying under a vacuum to produce the Chinese herbal medicine extract. Therein, the Chinese herbal medicine extract may be produced as freeze-dried powder for easy storage and further formulation.

In the present embodiment, the Chinese herbal medicine extract may be formulated as aqueous solutions of different concentration in a solvent. The solvent may be water, RO water, DI water, ethanol, methanol, isopropanol, or any other suitable, low-irritation solvent.

Analysis has been performed for the Chinese herbal medicine extract of the first embodiment of the present invention. In the present embodiment, the active ingredient of the Chinese herbal medicine extract contained agarwood, Chinese honeylocust fruit, Chinese honeylocust spine, cinnamon leaf, and camphor leaf in a weight ratio of 1:1:1:1:1. The analysis evaluated capability of the Chinese herbal medicine extract in intervening binding between SARS-COV-2 spike proteins and hACE2 (Human Angiotensin-converting Enzyme 2) and inhibiting activity of SARS-CoV-2 3CL pro protease. Each of the analysis results was the mean±the standard deviation over 50 experiments.

### 1. Analysis of Intervention in Binding for Complete Sequences of SARS-CoV-2 Spike Proteins

For SARS-COV-2 to enter human cells, its spike proteins play an important role. In the present embodiment, the enzyme-linked immunosorbent assay (ELISA) was used to evaluate capability of the disclosed Chinese herbal medicine extract in terms of inhibition and intervention with respect to binding of the complete sequences of spike proteins of SARS-COV-2 (the Wuhan strain) to hACE2. In the experiment, the used hACE2 was an in-vitro synthesized protein.

The ELISA experiment was conducted as described below.

First, 100 μL of a pH-9.6 spike protein solution (500 ng/mL; CAT. GTX135972-pro, GeneTex, Taipei, Taiwan, China) diluted with 15 mM of sodium carbonate and 35 mM of sodium hydrogen carbonate was placed into a 96-well microplate and the microplate was sealed and let stand at 4° C. overnight. Afterward, the 96-well microplate with spike protein solution was washed using a wash buffer solution made of a 0.05% (v/v) phosphate buffer solution and Tween-20 for three times for removing the part of the spike protein solution not attaching to the 96-well microplate.

Then 300 μL of an ELISA blocking solution made of a wash buffer solution containing 0.5% (w/v) fetal bovine serum was added into the 96-well microplate and cultivation was performed at 37° C. for 1.5 hours. After three times of wash, 100 μL of the disclosed Chinese herbal medicine extract at different concentrations (0 to 100000 μg/mL) or 100 μL of an inhibitor (10 ug/mL; cat. GTX635791, GeneTex, Taipei, Taiwan, China) was added into the 96-well microplate, and cultivation was performed at 37° C. for 1 hour before the 96-well microplate was washed with the wash buffer solution for three times.

Afterward, 100 μL of ELISA signal amplifier Streptavidin-HRP (100 ng/mL; cat. GTX30949, GeneTex, Taipei, Taiwan, China) was added and cultivated at 37° C. for 1 hour.

At last, in each well of the 96-well microplate, 200 μL of TMB substrate was added and cultivated in darkness at 37° C. for 1 hour. After completion of the reaction, 50 μL of a stop solution was added, and absorbance was measured at 450 nm using a microplate reader.

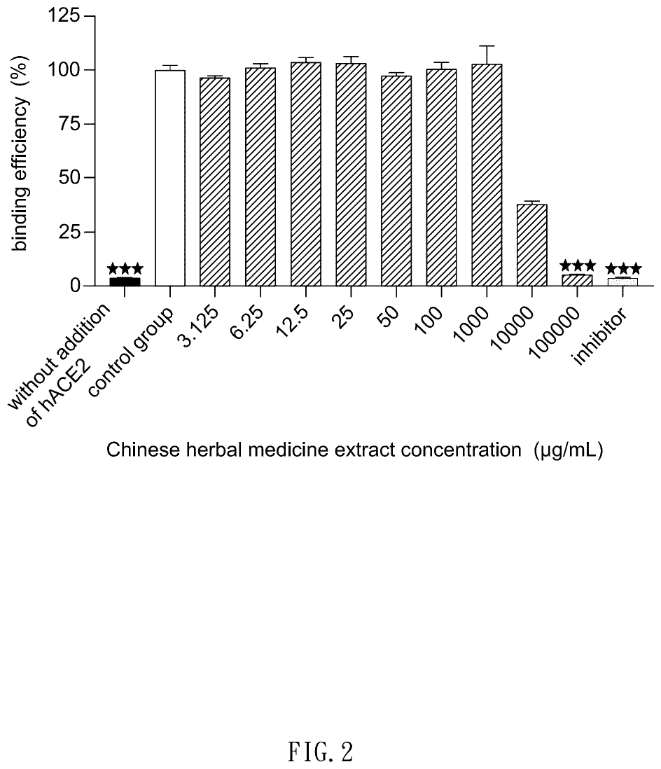

FIG. 2 shows intervention in binding of SARS-COV-2 spike proteins to hACE2 achieved by the Chinese herbal medicine extract of the present invention and an inhibitor (i.e., the RBD antibody), respectively. As shown, without addition of hACE2, only the background signals related to the SARS-COV-2 spike protein coating were detected. The control group was prepared by adding hACE2 into the SARS-COV-2 spike protein coating. The SARS-COV-2 spike protein coating with hACE2 added then generated strong colorimetric signals and had the binding efficiency increased to 100%. The experimental groups were prepared by adding different concentrations of the Chinese herbal medicine extract to intervene binding between SARS-COV-2 spike proteins and hACE2.

As observed, when 10000 μg/mL or more of the Chinese herbal medicine extract was added, the binding efficiency of SARS-COV-2 spike proteins to hACE2 decreased to less than 50%. Particularly, when the concentration of the Chinese herbal medicine extract was 100000 μg/mL, the binding efficiency of SARS-COV-2 spike proteins to hACE2 decreased to 4%, which is comparable to the inhibitor (i.e. the RBD antibody) in terms of inhibiting binding of SARS-COV-2 spike proteins to hACE2.

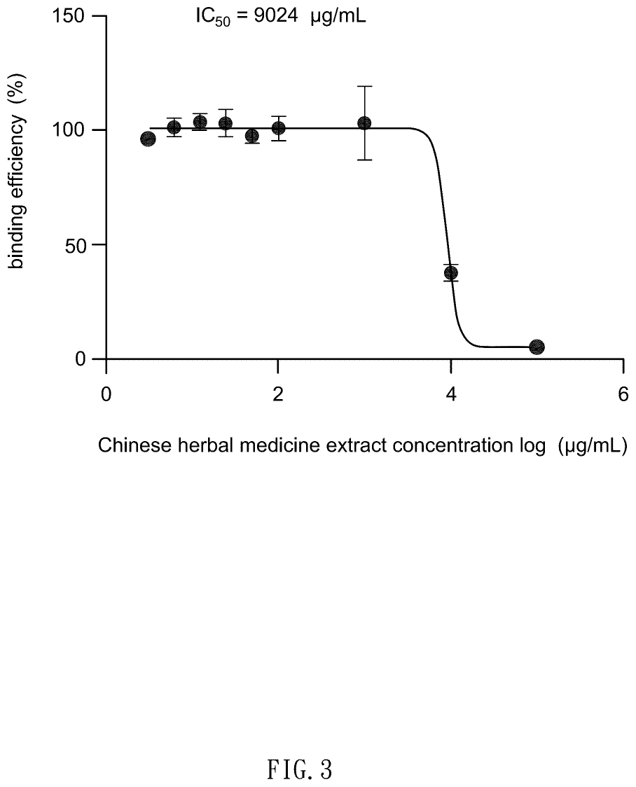

Nonlinear regression analysis was also performed to assert the impact of the disclosed Chinese herbal medicine extract on the binding efficiency of SARS-COV-2 spike proteins to hACE2. After 50 times of experiments, the Chinese herbal medicine extract of the present invention had a half-maximal inhibitory concentration IC50 of 7210˜10850 μg/mL. The preferred average of the half-maximal inhibitory concentration IC50 was 9024 μg/mL, as shown in FIG. 3. The results demonstrate that the disclosed Chinese herbal medicine extract was dose-dependently effective in inhibiting binding efficiency of spike proteins to hACE2.

A neutralization test was further performed for the Chinese herbal medicine extract and SARS-COV-2 pseudovirus. For the test, the used solvent was DI water, and the concentration of the Chinese herbal medicine extract was 250 mg/mL. Other conditions are listed below.

| | |
|---|---|
| Cell Strain | HeLa Expressing SmBiT-ACE2 |
| Repetition | 3 |
| Serial dilution | 1x for 5-fold dilution |
| | 7x for 2-fold dilution |
| Cultivation | 2 days |
| period | |
| Control group | PBS |
| Testing reagent | NanoGlo live cell assay system |
| Max testing | 1 mg/mL (1/250 dilution) |
| concentration | |

HeLa expressing SmBiT-ACE2 cells, SARS-COV-2 pseudovirus, and the Chinese herbal medicine extract of different concentrations were cultivated together for 6 hours. A PBS solution of equal amount was used for the control group. After 24-hour cultivation, the cells were washed twice using a PBS solution. At last, a bioluminescence test was conducted using NanoLuc.

The results of the test are shown in the table below as neutralization data with respect to the Chinese herbal medicine extract and SARS-COV-2 pseudovirus. As shown, the minimal dose of the Chinese herbal medicine extract for blocking infection of SARS-COV-2 to the cells was 50 μg/mL, with a blocking rate as high as 95%

| Chinese | | | | |
|---|---|---|---|---|
| herbal | | | | |
| medicine | Inhibition rate of viral entry (%) | | | |
| extract | Experiment- | Experiment- | Experiment- | |
| μg/mL | 1 | 2 | 3 | Average |
| 1000 | 100.0 | 100.0 | 100.0 | 100.0 |
| 200 | 100.0 | 100.0 | 100.0 | 100.0 |
| 100 | 99.8 | 100.0 | 99.9 | 99.9 |
| 50 | 95.9 | 97.2 | 91.8 | 95.0 |
| 25 | 39.4 | 56.8 | 41.1 | 45.8 |
| 12.5 | 28.4 | 30.9 | 25.7 | 28.3 |
| 6.25 | 5.1 | −3.9 | 3.9 | 1.6 |
| 3.125 | −12.0 | 5.6 | 0.7 | −1.9 |
| 1.5625 | −8.7 | 7.7 | −18.0 | −6.3 |

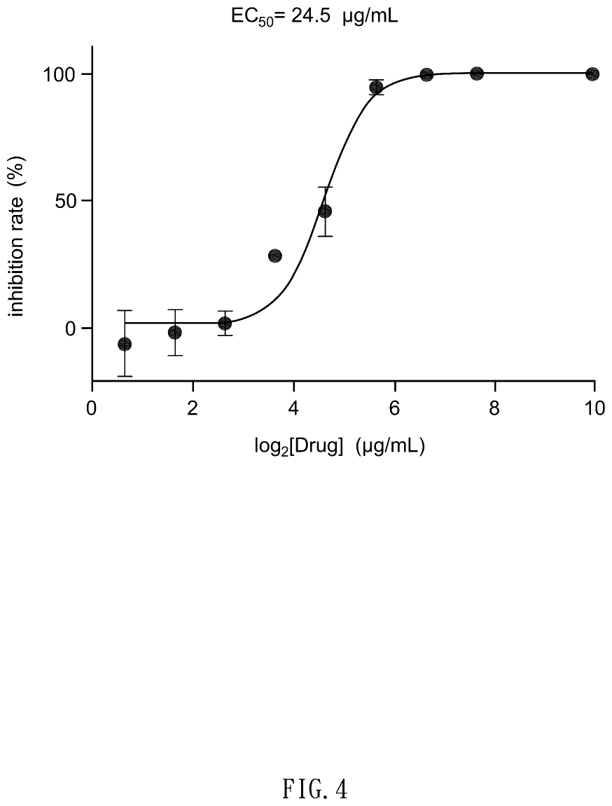

Moreover, FIG. 4 shows analysis of the half-maximal effective concentration EC50 of the Chinese herbal medicine extract with respect to binding between SARS-COV-2 pseudovirus and HeLa SmBiT-ACE2 expressing cells. As shown, the Chinese herbal medicine extract exhibited dose-dependently neutralizing activity against SARS-COV-2 pseudovirus, with the half-maximal effective concentration EC50 (or the concentration for 50% of maximal effect) ranging between 20 and 30 μg/mL. The preferred average of the half-maximal effective concentration EC50 was 24.5 μg/mL.

### 2. Analysis of Inhibition of Activity of SARS-CoV-2 3CLpro Protease

SARS-CoV-2 3CLpro protease is an enzyme participating in synthesis and replication of viral RNA. In the present embodiment, the capability of the disclosed Chinese herbal medicine extract to inhibit activity of SARS-CoV-2 3CLpro protease was evaluated using fluorescence resonance energy transfer (FRET).

The FRET-based evaluation involved the following steps.

First, a modified peptide DABCYL-TSAVLQSGFRKMG-EDANS (Genomics, Taiwan, China) was dissolved in water and cultivated together with SARS CoV-2 3CLpro.

Then a multimode microplate reader (SPARK® Multimode Microplate Reader, TECAN) was used to conduct the experiment at 37° C. so as to ensure activity of proteins, and the EDANS fluorescence intensity upon hydrolysis of the modified peptide was taken as the time function. Therein, the emission wavelengths were 340 nm and 490 nm, and the bandwidths were 9 nm and 15 nm, respectively.

Before testing, the samples were checked to ensure that their emission wavelengths would not overlap the emission spectrum of EDANS at 340 nm. At last, the impact of the disclosed Chinese herbal medicine extract at different concentrations (0 to 31.25 μg/mL) on the activity of SARS-CoV-2 3CLpro protease was measured and statistically analyzed using GraphPad Prism 7.03 (GraphPad Software, San Diego, CA, USA).

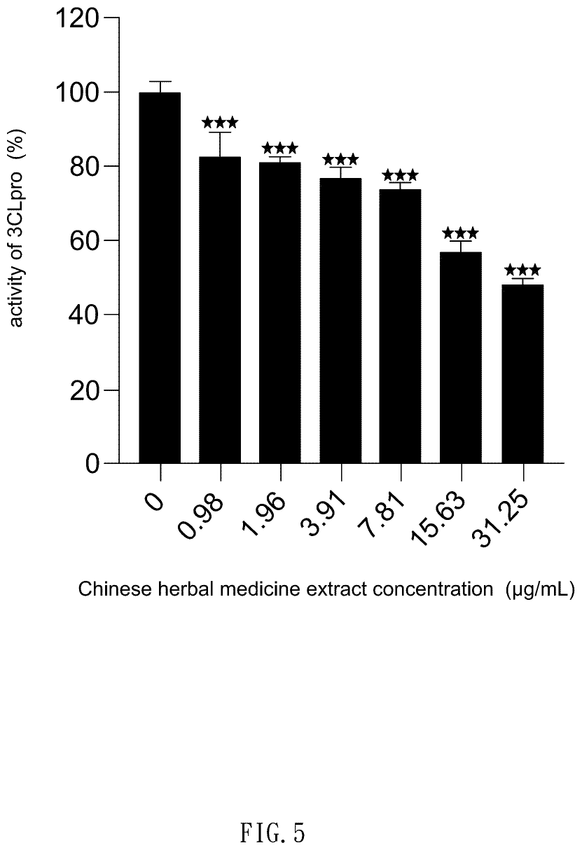

FIG. 5 shows inhibition of the activity of SARS-CoV-2 3CLpro protease by the Chinese herbal medicine extract of the present invention. As observed, after use of 0.98 μg/mL of the Chinese herbal medicine extract, the relative activity of SARS-CoV-2 3CLpro decreased to about 80% and after use of 7.81 μg/mL of the Chinese herbal medicine extract, the relative activity of SARS-CoV-2 3CLpro decreased to about 70%. As the concentration of the Chinese herbal medicine extract increased to 15.63˜31.25 μg/mL, the relative activity of SARS-CoV-2 3CLpro was further suppressed, decreasing to about 60% and 50%.

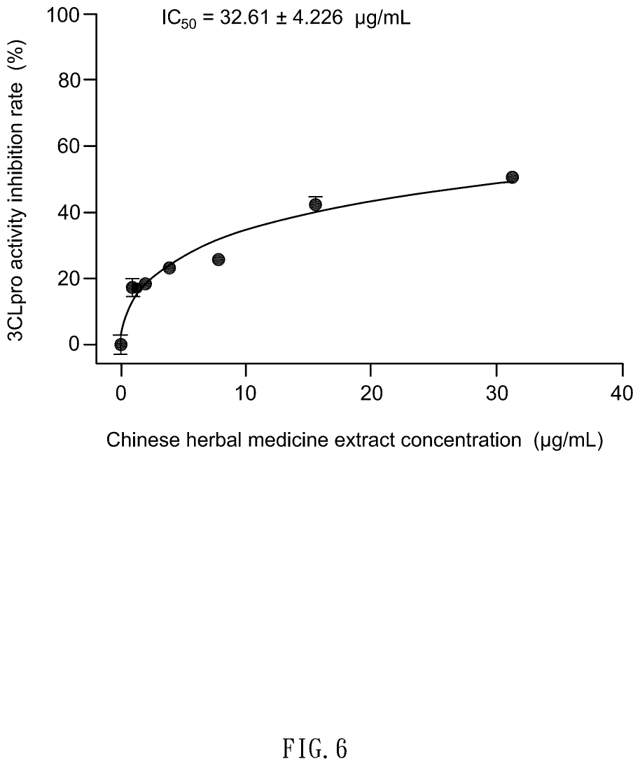

After 50 experiments, the half-maximal inhibitory concentration IC50 of the disclosed Chinese herbal medicine extract for inhibiting the activity of SARS-CoV-2 3CLpro protease was 26˜40 μg/mL, with the preferred average of the half-maximal inhibitory concentration IC50 being 32.61±4.226 μg/mL, as shown in FIG. 6. The results demonstrated that the Chinese herbal medicine extract of the present invention dose-dependently decreased the relative activity of SARS-CoV-2 3CLpro, suggesting the potential of the disclosed Chinese herbal medicine extract for treating SARS-CoV-2.

The analysis results about the Chinese herbal medicine extract of the second embodiment of the present invention are disclosed below. In the present embodiment, the active ingredient of the Chinese herbal medicine extract was cinnamon leaf. The analysis evaluated the capability to inhibit the activity of SARS-CoV-2 3CLpro protease. Since the steps have been detailed in the first embodiment, repeated description is omitted herein. Each of the analysis results was the mean±the standard deviation over 50 experiments.

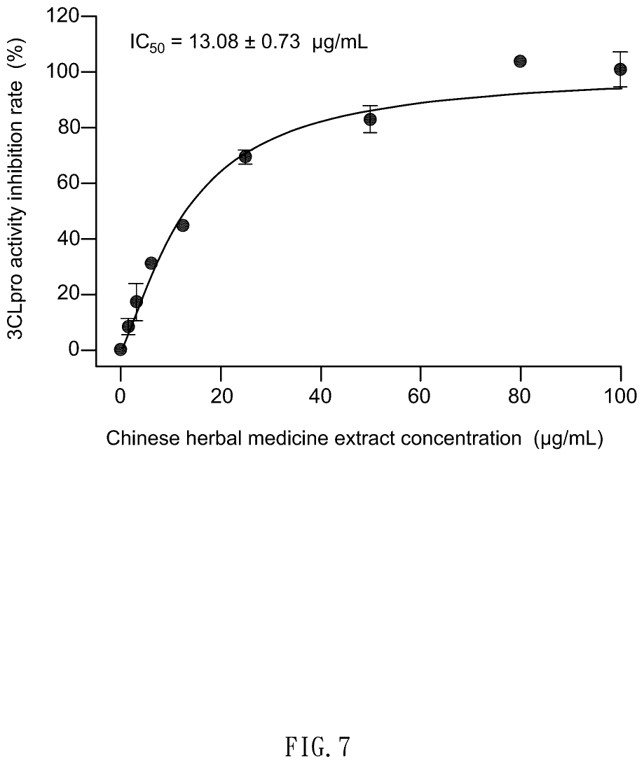

As shown in FIG. 7, according to the 50 experiments, the half-maximal inhibitory concentration IC50 of the Chinese herbal medicine extract of the present invention for inhibiting the activity of SARS-CoV-2 3CLpro protease was 10˜15 μg/mL, with the preferred average of the half-maximal inhibitory concentration IC50 being 13.08±0.73 μg/mL.

Impressively, in the present embodiment, the Chinese herbal medicine extract at 25 μg/mL and 50 μg/mL inhibited the activity of SARS-CoV-2 3CLpro protease by 69% and 83%, respectively, and the Chinese herbal medicine extract at 80 μg/mL completely inhibited the activity of SARS-CoV-2 3CLpro protease.

As demonstrated in the foregoing embodiments, the cinnamon leaf extract excelled in inhibiting the activity of SARS-CoV-2 3CLpro protease. Based on this, the present invention when implemented may use active ingredients of different compositions and with different proportions of materials to achieve desired pharmacological effects. In other words, the disclosed medication may be manufactured using any simple formula or complex formula suitable for certain coronavirus-related symptoms or diseases so as to enhance or expand therapeutical effects according to severity, thereby achieving the optimal therapeutic efficacy.

For example, for patients in the early stage of coronavirus-infected pneumonia, in view that the viral load in the patients is high, the medication may be such made that the share of cinnamon leaf is relatively high to effectively inhibit virus and reduce the binding efficiency of the virus. The weight ratio of cinnamon leaf, agarwood, Chinese honeylocust fruit, Chinese honeylocust spine and camphor leaf may be 1.5:1:1:1:1, or 2:1:1:1:1, or 2.5:1:1:1:1, or 3:1:1:1:1, or 3.5:1:1:1:1, or 4:1:1:1:1, or 4.5:1:1:1:1, or 5:1:1:1:1.

As also discussed previously, agarwood is effective in regulating qi-flow for relieving pain, downbearing counterflow for regulating spleen and stomach, warming kidney for improving qi reception, dredging channels for tranquillization, and sedation as well as analgesia. Chinese honeylocust fruit is effective in dispelling stubborn phlegm, breaking block and freeing orifices, relieving rheumatic pains, and destroying parasites. Chinese honeylocust spine is effective in dispersing swelling, resolving toxin, expelling pus, and destroying parasites. Camphor leaf is effective in relieving rheumatic pains, eliminating dampness, resolving toxin, and destroying parasites. Therefore, for patients in middle or late stages of coronavirus-infected pneumonia, since the viral load in the patients has been decreased, the share of cinnamon leaf can be reduced while the shares of agarwood, Chinese honeylocust fruit, Chinese honeylocust spine, and camphor leaf can be increased to enhance desired effects such as relieving pains, dispelling phlegm, dispersing swelling, and eliminating dampness.

Furthermore, the Chinese herbal medicine extract of the present invention may be used to manufacture a medication for treating a coronavirus-related symptom or disease. Therein, the medication comprises the Chinese herbal medicine extract of the present invention and a pharmaceutically acceptable carrier.

According to the present invention, the “carrier” refers to any drug carrier conventionally used for pharmaceutical purposes, such as a filling agent, a binding agent, a diluting agent, an excipient, a receptor or the like, as known to people skilled in the art.

In the present embodiment, the medication may be formulated as a mouthwash, an oral/nasal inhaler, or an oral capsule, without limitation. Therein, use of a mouthwash containing the Chinese herbal medicine extract helps to bind and remove coronavirus remaining in the oral cavity and oropharynx. Use of an oral/nasal inhaler containing the Chinese herbal medicine extract allows the Chinese herbal medicine extract to be absorbed through nasal or pulmonary capillaries so as to kill coronavirus. Use of an oral capsule containing the Chinese herbal medicine extract allows the Chinese herbal medicine extract to be directly absorbed by the human body through the stomach, thereby killing coronavirus.

According to the present invention, the dosage of the medication manufactured using the Chinese herbal medicine extract may be determined by competent medical professionals according to the patient's age, body weight, health state, and disease progression with reference to common knowledge in the art. The medication manufactured using the Chinese herbal medicine extract may be administrated alone or together with other drugs, with the administration course implemented by physicians or competent professionals according to pharmaceutical conventions.

Thereby, administration of the medication containing the Chinese herbal medicine extract is effective in intervening binding of spike proteins of SARS-CoV-2 coronavirus to hACE2 and inhibiting the activity of 3CLpro protease of SARS-CoV-2 coronavirus, so as to achieve treatment of coronavirus-related symptoms or diseases.

The present invention has been described with reference to the preferred embodiments and it is understood that the embodiments are not intended to limit the scope of the present invention. Moreover, as the contents disclosed herein should be readily understood and can be implemented by a person skilled in the art, all equivalent changes or modifications which do not depart from the concept of the present invention should be encompassed by the appended claims.

1.-16. (canceled)

17. A Chinese herbal medicine extract, comprising an active ingredient that contains any one of or any combination of agarwood, Chinese honeylocust fruit, Chinese honeylocust spine, cinnamon leaf, and camphor leaf.

18. The Chinese herbal medicine extract of claim 17, wherein the active ingredient contains agarwood, Chinese honeylocust fruit, Chinese honeylocust spine, cinnamon leaf, and camphor leaf in a weight ratio of 1˜5:1˜5:1˜5:1˜5:1˜5.

19. The Chinese herbal medicine extract of claim 18, wherein the weight ratio of agarwood, Chinese honeylocust fruit, Chinese honeylocust spine, cinnamon leaf, and camphor leaf is 1:1:1:1:1.

20. A method for preparing the Chinese herbal medicine extract of claim 17, comprising steps of:
extraction: mixing a raw material containing any one of or any combination of agarwood, Chinese honeylocust fruit, Chinese honeylocust spine, cinnamon leaf, and camphor leaf with ultrapure water in a weight ratio of between 1:5 and 1:10 to form a mixture, freezing and thawing the mixture for cycles, and extracting the mixture under heating to obtain a liquid extract, in which the mixture is extracted at an extraction temperature of between 90° C. and 110° C. for an extraction period of between 1.5 hours and 2.5 hours;
filtration: filtering the liquid extract; and
dehydration: freeze drying the liquid extract under a vacuum to obtain the Chinese herbal medicine extract.

21. The method of claim 20, wherein the raw material contains agarwood, Chinese honeylocust fruit, Chinese honeylocust spine, cinnamon leaf, and camphor leaf in a weight ratio of 1˜5:1˜5:1˜5:1˜5:1˜5.

22. The method of claim 21, wherein the weight ratio of agarwood, Chinese honeylocust fruit, Chinese honeylocust spine, cinnamon leaf, and camphor leaf is 1:1:1:1:1.

23. A method for treating a coronavirus-related symptom or disease, comprising administration of a medication manufactured with the Chinese herbal medicine extract of claim 17, wherein the active ingredient of the Chinese herbal medicine extract contains cinnamon leaf.

24. The method of claim 23, wherein the medication is used for intervening spike proteins of SARS-COV-2.

25. The method of claim 23, wherein the medication is used for inhibiting activity of SARS-CoV-2 3CLpro protease.

26. The method of claim 24, wherein the medication has a half-maximal effective concentration EC50 of 20˜30 μg/mL.

27. The method of claim 25, wherein the medication has a half-maximal inhibitory concentration IC50 of 10˜15 μg/mL.

28. The method of claim 23, wherein the medication is formulated as a mouthwash, an oral/nasal inhaler, or an oral capsule.

29. The method of claim 24, wherein the medication is formulated as a mouthwash, an oral/nasal inhaler, or an oral capsule.

30. The method of claim 25, wherein the medication is formulated as a mouthwash, an oral/nasal inhaler, or an oral capsule.
